(19) United States
(12) Patent Application Publication
(10) Pub. No.: US 2016/0226001 A1
(43) Pub. Date: Aug. 4, 2016

(54) Organic Electroluminescent Device

(71) Applicant: MERCK PATENT GMBH, Darmstadt (DE)

(72) Inventors: Amir Hossain Parham, Frankfurt Am Main (DE); Philipp Stoessel, Frankfurt Am Main (DE); Christof Pflumm, Darmstadt (DE); Anja Jatsch, Frankfurt Am Main (DE); Joachim Kaiser, Darmstadt (DE)

(73) Assignee: Merck Patent GmbH, Darmstadt (DE)

(21) Appl. No.: 15/021,125

(22) PCT Filed: Aug. 13, 2014
(86) PCT No.: PCT/EP2014/002225

§ 371 Date: Mar. 10, 2016

(30) Foreign Application Priority Data
Sep. 11, 2013 (EP) 13004412.6

(51) Int. Cl. H01L 51/00; C09K 11/02; H01L 51/50; C09K 11/06

(52) U.S. Cl. CPC H01L 51/0067; H01L 51/0072; H01L 51/0073; C09K 11/06; C09K 11/025; H01L 51/5004; C09K 2211/1029; C09K 2211/1007; H01L 51/5012; H01L 2251/552; H01L 51/56

(57) ABSTRACT

The invention relates to an organic electroluminescent device (OLED) containing an emitting layer which contains a compound having a small difference between the S₁ and the T₁ energy levels and to another compound of formula (I) or (II).

The present application relates to an organic electroluminescent device (OLED) comprising an emitting layer, where the emitting layer comprises a compound having a small difference between the energies of the S₁ and T₁ states and additionally a further compound of a formula (I) or (II).

In general, the term OLED is taken to mean an electronic device which comprises at least one organic material and which emits light on application of electrical voltage. The basic structure of OLEDs is known to the person skilled in the art and is described, inter alia, in U.S. Pat. No. 4,539,507, U.S. Pat. No. 5,151,629, EP 0676461 and WO 98/27136.

The energies of the S₁ and T₁ states of a compound are, for the purposes of the present application, defined as the energies that are obtained for the relevant states of the compound by quantum-chemical calculations. The S₁ state here is the energetically lowest excited singlet state, and the T₁ state is the energetically lowest triplet state. The precise way in which the quantum-chemical calculations are carried out is described in the working examples.

In the development of novel OLEDs, there is considerable interest in an improvement in the efficiency and operating voltage of the devices. Furthermore, there is interest in an improvement in the lifetime of the devices. Still furthermore, there is interest in the provision of OLEDs which are simple and inexpensive to produce and which can be built up, in particular, from inexpensive materials which can be prepared simply.

It is known from the prior art that OLEDs having very good efficiencies can be obtained with certain purely organic emitting compounds which do not phosphoresce, but instead fluoresce. For example, H. Uoyama et al., Nature 2012, 492, 234, discloses that OLEDs having external quantum efficiencies which are similar to or better than those which can be obtained with phosphorescent emitters can be obtained with carbazolylcyano-benzene compounds as emitting compounds.

The emitting compounds used in this publication have a small energy difference between the S₁ and T₁ states. The energy difference is preferably in the region of the thermal energy or less. The said publication describes the use of these emitting compounds in the emitting layer in combination with a further compound which represents a matrix material. The matrix material disclosed is, inter glia, the carbazole derivative CBP and the arylphosphine oxide PPT.

It is furthermore known from the prior art that the above-mentioned emitting compounds having a small energy difference between the S₁ and T₁ states can advantageously also be employed in combination with the arylsilane compound TBSi-F as matrix material (G. Mehes. Angew. Chem. Int. Ed. 2012, 51, 11311-11315).

In spite of these advances, there continues to be a need for improvement in the area of OLEDs, in particular in the area of compositions for the emitting layer of these devices. There is also considerable interest in the provision of alternative embodiments for the emitting layer of OLEDs which result in performance data of the OLEDs which are comparable to those in the prior art.

To this end, investigations have been conducted as part of the present application into which compounds are suitable as matrix materials for use in combination with the above-mentioned emitting compounds. To this end, a vast multiplicity of structural classes which are known to be suitable for use as functional materials in OLEDs is available to the person skilled in the art.

In these investigations, it has been found, surprisingly, that aryldibenzo-furan compounds and aryldibenzothiophene compounds having a certain structure are highly suitable for use as matrix materials in combination with the above-mentioned emitting compounds having a small energy difference between the S₁ and T₁ states. Inter alfa, very good values for the operating voltage U1000, the external quantum efficiency EQE, the lifetime and the roll-off (for explanation cf. working examples) are obtained here. Compared with electroluminescent devices comprising iridium or platinum complexes as emitters, a longer lifetime at elevated temperature is obtained in the case of the devices according to the invention.

The present application thus relates to an organic electroluminescent device

comprising an emitting layer which comprises a compound E and a compound M,
where compound E has a difference in value between the energies of the S₁ and T₁ states of at most 0.15 eV, and
where compound M conforms to a formula (I) or (ll)

for which:

Y is on each occurrence, identically or differently, O, S or Se;

Z is on each occurrence, identically or differently, CR¹, C or N, where Z is equal to C precisely if a group L¹ or L² is bonded to Z together with the group bonded thereto;

L¹, L² are on each occurrence, identically or differently, a single bond or a divalent group;

Ar¹ is on each occurrence, identically or differently, an aromatic or heteroaromatic ring system having 5 to 40 aromatic ring atoms, which may be substituted by one or more radicals R²;

R¹, R² are on each occurrence, identically or differently, H, O, F, C(═O)R³, CN, Si(R³)₃, N(R³)₂, P(═O)(R³)₂, OR³, S(═O)R³, S(═O)₂R³, a straight-chain alkyl or alkoxy group having 1 to 20 C atoms or a branched or cyclic alkyl or alkoxy group having 3 to 20 C atoms or an alkenyl or alkynyl group having 2 to 20 C atoms, where the above-mentioned groups may each be substituted by one or more radicals R³ and where one or more CH₂ groups in the above-mentioned groups may be replaced by —R³C═CR³—, —C≡C—, Si(R³)₂, C═O, C═NR³, —C(═O)O—, —C(═O)NR³—, NR³, P(═O)(R³), —O—, —S—, SO or SO₂, or an aromatic or heteroaromatic ring system having 5 to 30 aromatic ring atoms, which may in each case be substituted by one or more radicals R³, where two or more radicals R¹ or R² may be linked to one another and may form a ring;

R³ is on each occurrence, identically or differently, H, O, F, C(═O)R⁴, CN, Si(R⁴)₃, N(R⁴)₂, P(═O)(R⁴)₂, OR⁴, S(═O)R⁴, S(═O)₂R⁴, a straight-chain alkyl or alkoxy group having 1 to 20 C atoms or a branched or cyclic alkyl or alkoxy group having 3 to 20 C atoms or an alkenyl or alkynyl group having 2 to 20 C atoms, where the above-mentioned groups may each be substituted by one or more radicals R⁴ and where one or more CH₂ groups in the above-mentioned groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂, or an aromatic or heteroaromatic ring system having 5 to 30 aromatic ring atoms, which may in each case be substituted by one or more radicals R⁴, where two or more radicals R³ may be linked to one another and may form a ring;

R⁴ is on each occurrence, identically or differently, H, O, F or an aliphatic, aromatic or heteroaromatic organic radical having 1 to 20 C atoms, in which, in addition, one or more H atoms may be replaced by D or F; two or more substituents R⁴ here may be linked to one another and may form a ring;

n is equal to 0 or 1;

where the energies are determined by quantum-chemical calculation, as described in the working examples.

A divalent group in the sense of the present application is taken to mean any desired organic group which has two free bonds. This can be, for example, a heteroatom, a hydrocarbon chain, a hydrocarbon ring, or a stringing together or linking of a plurality of the above-mentioned units. The units here may be substituted or unsubstituted.

An aryl group in the sense of this invention contains 6 to 60 aromatic ring atoms; a heteroaryl group in the sense of this invention contains 5 to 60 aromatic ring atoms, at least one of which is a heteroatom. The heteroatoms are preferably selected from N, O and S. This represents the basic definition. If other preferences are indicated in the description of the present invention, for example with respect to the number of aromatic ring atoms or the heteroatoms present, these apply.

An aryl group or heteroaryl group here is taken to mean either a simple aromatic ring, i.e. benzene, or a simple heteroaromatic ring, for example pyridine, pyrimidine or thiophene, or a condensed (annellated) aromatic or heteroaromatic polycycle, for example naphthalene, phenanthrene, quino-line or carbazole. A condensed (annellated) aromatic or heteroaromatic polycycle in the sense of the present application consists of two or more simple aromatic or heteroaromatic rings condensed with one another.

An aryl or heteroaryl group, which may in each case be substituted by the above-mentioned radicals and which may be linked to the aromatic or heteroaromatic ring system via any desired positions, is taken to mean, in particular, groups derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindoie, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazoie, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the sense of this invention contains 6 to 60 C atoms in the ring system. A heteroaromatic ring system in the sense of this invention contains 5 to 60 aromatic ring atoms, at least one of which is a heteroatom. The heteroatoms are preferably selected from N, O and/or S, An aromatic or heteroaromatic ring system in the sense of this invention is intended to be taken to mean a system which does not necessarily contain only aryl or heteroaryl groups, but instead in which, in addition, a plurality of aryl or heteroaryl groups may be connected by a non-aromatic unit (preferably less than 10% of the atoms other than H), such as, for example, an sp³-hybridised C, Si, N or O atom, an sp²-hybridised C or N atom or an sp-hybridised C atom, Thus, for example, systems such as 9,9′-spirobifluorene, 9,9′-diarylfluorene, triarylamine, diaryl ether, stilbene, etc., are also intended to be taken to be aromatic ring systems in the sense of this invention, as are systems in which two or more aryl groups are connected, for example, by a linear or cyclic alkyl, alkenyl or alkynyl group or by a silyl group. Furthermore, systems in which two or more aryl or heteroaryl groups are linked to one another via single bonds are also taken to be aromatic or heteroaromatic ring systems in the sense of this invention, such as, for example, systems such as biphenyl, terphenyl or diphenyltriazine.

An aromatic or heteroaromatic ring system having 5-60 aromatic ring atoms, which may in each case also be substituted by radicals as defined above and which may be linked to the aromatic or heteroaromatic group via any desired positions, is taken to mean, in particular, groups derived from benzene, naphthalene, anthracene, benzanthracene, phenanthrene, benzophenanthrene, pyrene, chrysene, perylene, fluoranthene, naphthacene, pentacene, benzopyrene, biphenyl, biphenylene, terphenyl, terphenylene, quaterphenyl, fluorene, spirobifluorene, dihydrophenanthrene, dihydropyrene, tetrahydropyrene, cis- or trans-indenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthirnidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline , 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or combinations of these groups.

For the purposes of the present invention, a straight-chain alkyl group having 1 to 40 C atoms or a branched or cyclic alkyl group having 3 to 40 C atoms or an alkenyl or alkynyl group having 2 to 40 C atoms, in which, in addition, individual H atoms or CH₂ groups may be substituted by the groups mentioned above under the definition of the radicals, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl. An alkoxy or thioalkyl group having 1 to 40 C atoms is preferably taken to mean methoxy, trifluorornethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-tri-fluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio , hexenylthio, cyclohexenylthio, heptenylthio, cycloheptenyithio, _{oc}t_{eny}lthio, cyclooctenylthio, ethynylthio, propynylthio, butynylthio, pentynylthio, hexynylthio, heptynylthio or octynylthio.

The formulation that two or more radicals may form a ring with one another is intended for the purposes of the present application to be taken to mean, inter alia, that the two radicals are linked to one another by a chemical bond. Furthermore, however, the above-mentioned formulation is also intended to be taken to mean that, in the case where one of the two radicals represents hydrogen, the second radical is bonded to the position to which the hydrogen atom was bonded, with formation of a ring.

It is preferred for not more than two groups Z per ring to be equal to N, particularly preferably not more than one group Z per ring is equal to N.

According to a preferred embodiment, Z is on each occurrence, identically or differently, CR¹ or C, where Z is equal to C precisely if a group L¹ or L² is bonded to Z together with the group bonded thereto.

Y is preferably on each occurrence, identically or differently, O or S.

L¹ is preferably selected on each occurrence, identically or differently, from a single bond, Si(R²)₂, O, S or an alkylene group having 1 to 10 C atoms, in which one or more CH₂ groups may be replaced by Si(R²)₂, O, S, C═O, C═NR², C═O—O, C═O—NR², NR², P(═O)(R²), SO or SO₂ and which may be substituted by one or more radicals R², or an aromatic or heteroarornatic ring system having 5 to 30 aromatic ring atoms, which may in each case be substituted by one or more radicals R².

L¹ is particularly preferably selected on each occurrence, identically or differently, from a single bond, Si(R²)2 or an aromatic or heteroaromatic ring system having 5 to 30 aromatic ring atoms, which may be substituted by one or more radicals R²; L¹ is very particularly preferably selected on each occurrence, identically or differently, from a single bond or an aromatic or heteroaromatic ring system having 5 to 30 aromatic ring atoms, which may be substituted by one or more radicals R².

L² is preferably selected from a single bond, Si(R²)₂, O, S or an alkylene group having 1 to 10 C atoms, in which one or more CH₂ groups may be replaced by Si(R²)₂, O, S, C═O, C═NR², C═O—O, C═O—NR², NR², P(═O)(R²), SO or SO₂ and which may be substituted by one or more radicals R², or an aromatic or heteroaromatic ring system having 5 to 60 aromatic ring atoms, which may in each case be substituted by one or more radicals R².

L² is particularly preferably selected from a single bond or an aromatic or heteroaromatic ring system having 5 to 60 aromatic ring atoms, which may be substituted by one or more radicals R²; L² is very particularly preferably an aromatic or heteroarornatic ring system having 5 to 60 aromatic ring atoms, which may be substituted by one or more radicals R².

Ar¹ is preferably on each occurrence, identically or differently, an aromatic or heteroaromatic ring system having 5 to 18 aromatic ring atoms, particularly preferably 5 to 16 aromatic ring atoms, which may be substituted by one or more radicals R². Ar¹ is particularly preferably selected from phenyl, biphenyl, terphenyl, quaterphenyl, fluorenyl, spirobifluorenyl, indenofluorenyl, naphthyl, anthracenyl, phenanthrenyl, pyrenyl, fluoranthenyl, furanyl, benzofuranyl, isobenzofuranyl, dibenzofuranyl, thlophenyl, benzothiophenyl, isobenzothiophenyl, dibenzothiophenyl, pyrrolyl, indolyl, isoindolyl, carbazolyl, indolocarbazolyl, indenocarbazolyl, pyridyl, quinolinyl, isoquinolinyl, acridyl, pyrazolyl, imidazolyl, benzimidazolyl, pyridazyl, pyrimidyl, pyrazinyl and phenanthrolyl, each of which is optionally substituted by radicals R².

R¹ is preferably selected on each occurrence, identically or differently, from H, D, F, CN, Si(R³)₃, a straight-chain alkyl or alkoxy group having 1 to 10 C atoms or a branched or cyclic alkyl or alkoxy group having 3 to 10 C atoms, where the above-mentioned groups may each be substituted by one or more radicals R³ and where one or more CH₂ groups in the above-mentioned groups may be replaced by —C≡C—, —R³C═CR³—, Si(R³)₂, C═O, C═NR³, —NR³—, —O—, —S—, —C(═O)O— or —C(═O)NR³13 , or an aromatic or hetero-aromatic ring system having 5 to 20 aromatic ring atoms, which may in each case be substituted by one or more radicals R³, where two or more radicals Ri may be linked to one another and may form a ring.

R¹ is particularly preferably selected on each occurrence, identically or dif ferently, from H, F, CN, a straight-chain alkyl group having 1 to 10 C atoms or a branched or cyclic alkyl group having 3 to 10 C atoms, where the alkyl groups may each be substituted by one or more radicals R³, or an aromatic or heteroaromatic ring system having 5 to 20 aromatic ring atoms, which may in each case be substituted by one or more radicals R³.

R² is preferably selected on each occurrence, identically or differently, from H, D, F, CN, Si(R³)₃, a straight-chain alkyl or alkoxy group having 1 to 10 C atoms or a branched or cyclic alkyl or alkoxy group having 3 to 10 C atoms, where the above-mentioned groups may each be substituted by one or more radicals R³ and where one or more CH₂ groups in the above-mentioned groups may be replaced by —R³C═CR³—, Si(R³)₂, C═O, C═NR³, —NR³13 , —O—, —S—, —C(═O)O— or —C(═O)NR³13 , or an aromatic or hetero-aromatic ring system having 5 to 20 aromatic ring atoms, which may in each case be substituted by one or more radicals R³, where two or more radicals R² may be linked to one another and may form a ring.

R² is particularly preferably selected on each occurrence, identically or differently, from H, F, CN, a straight-chain alkyl group having 1 to 10 C atoms or a branched or cyclic alkyl group having 3 to 10 C atoms, where the alkyl groups may each be substituted by one or more radicals R³, or an aromatic or heteroaromatic ring system having 5 to 20 aromatic ring atoms, which may in each case be substituted by one or more radicals R³.

R³ is preferably selected on each occurrence, identically or differently, from H, D, F, CN, Si(R⁴)₃, a straight-chain alkyl or alkoxy group having 1 to 10 C atoms or a branched or cyclic alkyl or alkoxy group having 3 to 10 C atoms, where the above-mentioned groups may each be substituted by one or more radicals R⁴ and where one or more CH₂ groups in the above-mentioned groups may be replaced by —C≡C—, —R⁴C═CR⁴—, Si(R⁴)₂, C═O, C═NR⁴, —NR⁴—, —O—, —S—, —C(═O)O— or —C(═O)NR⁴—, or an aromatic or hetero-aromatic ring system having 5 to 20 aromatic ring atoms, which may in each case be substituted by one or more radicals R⁴, where two or more radicals R³ may be linked to one another and may form a ring.

R³ is particularly preferably selected on each occurrence, identically or differently, from H, F, CN, a straight-chain alkyl group having 1 to 10 C atoms or a branched or cyclic alkyl group having 3 to 10 C atoms, where the alkyl groups may each be substituted by one or more radicals R⁴, or an aromatic or heteroaromatic ring system having 5 to 20 aromatic ring atoms, which may in each case be substituted by one or more radicals R⁴.

In general and for all preferred embodiments of the formula (I), the bonding positions of L¹ can be in the positions selected from positions 1, 2, 3, 4, 6, 7, 8 and 9 of the central skeleton, as shown below.

In general and for all preferred embodiments of the formula (II), the bonding positions of L² can be in the positions selected from positions 1, 2, 3 and 4 of the central skeleton, as shown below,

Preferred embodiments of the formula (I) conform to the following formulae (I-1) to (I-4)

where the groups occurring are defined as above.

The groups occurring in formulae (I-1) to (I-4) preferably correspond to their preferred embodiments indicated above.

Preferred embodiments of formulae (I-1) and (I-3) are the following formulae (I-1-1) and (I-1-2) and (I-3-1) and (I-3-2):

where:

V is on each occurrence, identically or differently, CR², C or N, where V is equal to C precisely if a group L¹ is bonded thereto, and where the proviso applies that at least one group V is equal to N;

Z¹ is on each occurrence, identically or differently, CR², C or N, where Z¹ is equal to C precisely if a group L¹ is bonded thereto;

Ar² is a condensed aryl group having 10 to 14 aromatic ring atoms, which may be substituted by one or more radicals R²;

and where the other groups occurring are defined as above.

The groups occurring in formulae (I-1-1) to (I-1-2) and (I-3-1) to (I-3-2) preferably correspond to their preferred embodiments indicated above.

It is furthermore preferred for Z¹ to be equal to CR² or C, where Z¹ is equal to C precisely if a group L¹ is bonded thereto.

It is furthermore preferred for precisely 1, 2 or 3 groups V in the ring to be equal to N, and for the remaining groups V to be equal to CR² or C. It is preferred here for not more than two adjacent groups V in the ring to be equal to N, particularly preferably for no adjacent groups V in the ring to be equal to N.

It is furthermore preferred for L¹ in formulae (I-1-1) and (I-1-2) and (I-3-1) and (I-3-2) to be a single bond or a phenyl group or biphenyl group which is optionally substituted by radicals R².

Preferred embodiments of formulae (I-2) and (I-4) are the following formulae (I-2-1) to (I-2-4) and (I-4-1) to (I-4-4):

where:

V is on each occurrence, identically or differently, CR², C or N, where

V is equal to C precisely if a group L¹ is bonded thereto, and where the proviso applies that at least one group V is equal to N;

Z¹ is on each occurrence, identically or differently, CR², C or N, where Z¹ is equal to C precisely if a group L¹ is bonded thereto;

Ar^{e} is a condensed aryl group having 10 to 14 aromatic ring atoms, which may be substituted by one or more radicals R²;

and where the other groups occurring are defined as above.

The groups occurring in formulae (I-2-1) to (I-2-4) and (I-4-1) to (l-4-4) preferably correspond to their preferred embodiments indicated above,

It is furthermore preferred for Z¹ to be equal to CR² or C, where Z¹ is equal to C precisely if a group L¹ is bonded thereto.

It is furthermore preferred for precisely 1, 2 or 3 groups V in the ring to be equal to N, and for the remaining groups V to be equal to CR² or C. It is preferred here for not more than two adjacent groups V in the ring to be equal to N, particularly preferably for no adjacent groups V in the ring to be equal to N.

It is particularly preferred for formulae (I-2-2), (I-2-3), (I-4-2) and (I-4-3) for at least one group R² as substituent of a group Zi to represent a carbazole group which is optionally substituted by radicals R³.

It is furthermore preferred for Ar² to be a phenanthrenyl group, which may be substituted by one or more radicals R².

It is furthermore preferred for formulae (I-2-1) to (I-2-4) and (I-4-1) to (I4-4) for L¹ to be a single bond, a group Si(R²)₂ or a phenyl group or biphenyl group which is optionally substituted by radicals R².

Preferred embodiments of the formula (II) conform to the following formulae (II-1) to (II-3)

where the groups occurring are defined as above.

The groups occurring in formulae (II-1) to (II-3) preferably correspond to their preferred embodiments indicated above.

The group L² in formulae (II-1) to (II-3) is preferably selected from a single bond or a unit of the formula (L2)

where:

Ar³ is on each occurrence, identically or differently, an ary ene or heteroarylene group having 6 to 14 aromatic ring atoms, which may be substituted by one or more radicals R²; and

k is equal to 1, 2, 3 or 4.

Ar³ here is preferably selected from phenyl, pyridyl, pyrimidyl, naphthyl, phenanthrenyl, quinollnyl, carbazolyl, dibenzofuranyl and dibenzothiophenyl.

k here is preferably equal to 1, 2 or 3.

The following compounds are examples of compounds M of the formula (I) or (II):

Compound M of the formulae (I) and (II) can be prepared by known processes of organic synthesis, for example bromination, Buchwald coupling and Suzuki coupling.

Synthetic processes for the preparation of compound M of the formula (I) or (II) are described in detail, for example, in WO 2012/145173, WO 2012/048266, WO 2011/137072, EP 2551932, US 2009/0030202 and WO 2011/057706.

The organic electroluminescent device according to the invention is described in greater detail below.

In a preferred embodiment of the invention, compound M of the formula (I) or (II) is the matrix material in the emitting layer, and compound E is the emitting compound. Emitting compound is taken to mean the compound whose emission from the emitting layer during operation of the device is observed. In accordance with the invention, compound M of the formula (I) or (II) does not contribute or does not contribute significantly to the emission from the emitting layer.

In a preferred embodiment of the invention, the emitting layer essentially consists of compound M of the formula (I) or (II) and compound E. The emitting layer particularly preferably consists exclusively of compound M of the formula (I) or (II) and compound E.

Compound E is preferably present in the emitting layer in a significantly higher proportion than compound M. The proportion of compound M is preferably between 80% and 99%, particularly preferably between 90 and 98% and very particularly preferably between 93 and 97%. The proportion of compound E is preferably between 1% and 20%, particularly preferably between 2 and 10% and very particularly preferably between 3 and 7%.

The specifications of proportions in % here are taken to mean the proportion in per cent by volume in the case of compounds applied from the gas phase, and the proportion in per cent by weight in the case of compounds applied from solution.

It is furthermore preferred for the energy of the T₁ state of compound M of the formula (I) or (H) (T₁(M)) to be a maximum of 0.1 eV lower than the energy of the T₁ state of compound E (T₁(E)). T₁(M) is particularly preferably ≧T₁(E). Very particularly preferably: T₁(M)−T₁(E)≧0.1 eV, most preferably T₁(M)−T₁(E)≧0.2 eV. The energies of the T₁ states here are determined by quantum-chemical calculation, as described in the working examples.

Compound E is preferably an organic compound. An organic compound in the sense of the present invention is a carbon-containing compound which contains no metals. In particular, the organic compound is built up from the elements C, H, D, B, Si, N, P, O, S, F, Cl, Br and I.

Furthermore preferably, compound E is a luminescent compound. A luminescent compound in the sense of the present invention is a compound which is capable of emitting light at room temperature on optical excitation in an environment as is present in the organic electroluminescent device. The compound here preferably has a luminescence quantum efficiency of at least 40%, particularly preferably at least 50%, very particularly preferably at least 60% and especially preferably at least 70%. The luminescence quantum efficiency here is determined in a layer in a mixture with the matrix material, as is to be used in the organic electroluminescent device. The way in which the determination of the luminescence quantum yield is carried out for the purposes of the present invention is described in the example part.

It is furthermore preferred for compound E to have a short decay time. The decay time here is preferably ≦50 μs. The way in which the determination of the decay time is carried out for the purposes of the present invention is described in the example part.

The energies of the lowest excited singlet state (S₁) and the lowest triplet state (T₁) are determined by quantum-chemical calculation, The way in which this determination is carried out for the purposes of the present invention is described in the example part.

The difference in value between the energies of the S₁ and T₁ states of compound E is, in accordance with the invention, at most 0.15 eV. The difference in value is preferably ≦0.10 eV, particularly preferably ≦0.08 eV, very particularly preferably ≦0.05 eV.

Compound E is preferably an aromatic compound which contains both at least one donor substituent and also at least one acceptor substituent, where the LUMO and the HOMO of the compound only overlap spatially to a slight extent. What is understood under donor or acceptor substituent is known in principle to the person skilled in the art. Suitable donor substituents are, in particular, diarylamino and diheteroarylamino groups as well as carbazole groups and carbazole derivatives, each of which is preferably bonded to the aromatic compound via N. These groups may also be substituted further here. Suitable acceptor substituents are, in particular, cyano groups and electron-deficient heteroaryl groups, which may also be substituted further.

The following preferably applies to the LUMO of compound E (LUMO(E)) and the HOMO of compound M (HOMO(M)):

LUMO(E)−HOMO(M)>S₁(E)−0.4 eV;

particularly preferably:

LUMO(E)−HOMO(M)>S₁(E)−0.3 eV;

and very particularly preferably:

LUMO(E)−HOMO(M)>S₁(E)−0.2 eV.

S₁(E) here is the first excited singlet state S₁ of compound E, The said HOMO and LUMO energies LUMO(E) and HOMO(M) are determined here by quantum-chemical calculations, as described in the working examples.

Examples of compounds E for use in the device according to the invention are shown in the following table.

The organic electroluminescent device comprises cathode, anode and emitting layer. Apart from these layers, it may also comprise further layers, for example hole-injection layers, hole-transport layers, hole-blocking layers, electron-transport layers, electron-injection layers, exciton-blocking layers, electron-blocking layers and/or charge-generation layers. It preferably comprises one or more hole-transport layers, which are arranged between anode and emitting layer, and one or more electron-transport layers, which are arranged between cathode and emitting layer.

All materials which are known for this use from the prior art can be used in the further layers of the organic electroluminescent device according to the invention, in particular in the hole-injection and -transport layers and in the electron-injection and -transport layers.

The hole-transport layers here may also be p-doped and the electron-transport layers may also be n-doped. A p-doped layer is taken to mean a layer in which free holes are generated by a p-dopant and their conductivity is thus increased. A comprehensive discussion of doped transport layers in OLEDs can be found in Chem. Rev. 2007, 107, 1233. The p-dopant is particularly preferably capable of oxidising the hole-transport material in the hole-transport layer, i.e. has a sufficiently high redox potential, in particular a higher redox potential than the hole-transport material. Suitable p-dopants are in principle all compounds which are electron-acceptor compounds and are able to increase the conductivity of the organic layer by oxidation of the hole-transport material. The person skilled in the art will be able to identify suitable compounds without major effort on the basis of his general expert knowledge. Particularly suitable dopants are the compounds disclosed in WO 2011/073149, EP 1968131, EP 2276085, EP 2213662, EP 1722602, EP 2045848, DE 102007031220, U.S. Pat. No. 8,044,390, U.S. Pat. No. 8,057,712, WO 2009/003455, WO 2010/094378, WO 2011/120709 and US 2010/0096600.

The cathode of the organic electroluminescent device preferably comprises metals having a low work function, metal alloys or multilayered structures comprising various metals, such as, for example, alkaline-earth metals, alkali metals, main-group metals or lanthanoids (for example Ca, Ba, Mg, Al, In, Mg, Yb, Sm, etc.), Also suitable are alloys of an alkali metal or alkaline-earth metal and silver, for example an alloy of magnesium and silver. In the case of multilayered structures, further metals which have a relatively high work function, such as, for example, Ag or Al, can also be used in addition to the said metals, with combinations of the metals, such as, for example, Ca/Ag, Mg/Ag or Ba/Ag, then generally being used. It may also be preferred to introduce a thin interlayer of a material having a high dielectric constant between a metallic cathode and the organic semiconductor. Suitable for this purpose are, for example, alkali-metal or alkaline-earth metal fluorides, but also the corresponding oxides or carbonates (for example LiF, Li₂O, BaF₂, MgO, NaF, CsF, Cs₂CO_{3,} etc). Lithium quino-linate (LiQ) can furthermore be used for this purpose. The layer thickness of this layer is preferably between 0.5 and 5 nm.

The anode preferably comprises materials having a high work function. The anode preferably has a work function greater than 4.5 eV vs. vacuum.

Suitable for this purpose are on the one hand metals having a high redox potential, such as, for example, Ag. Pt or Au. On the other hand, metal/metal oxide electrodes (for example AliNi/NiOₓ, AliPtOₓ) may also be preferred. For some applications, at least one of the electrodes must be transparent or partially transparent in order to facilitate either irradiation of the organic material (organic solar cell) or the coupling-out of light (OLEDs, O-lasers). Preferred anode materials here are conductive mixed metal oxides. Particular preference is given to indium tin oxide (ITO) or indium zinc oxide (IZO). Preference is furthermore given to conductive, doped organic materials, in particular conductive, doped polymers. The anode may furthermore also consist of a plurality of layers, for example an inner layer of ITO and an outer layer of a metal oxide, preferably tungsten oxide, molybdenum oxide or vanadium oxide.

The device is correspondingly (depending on the application) structured, provided with contacts and finally sealed in order to exclude damaging effects of water and air.

In a preferred embodiment, the organic electroluminescent device is characterised in that one or more layers are applied by means of a sublimation process, in which the materials are applied by vapour deposition in vacuum sublimation units at an initial pressure of less than 10⁻⁵ mbar, preferably less than 10⁻⁶ mbar. However, it is also possible here for the initial pressure to be even lower, for example less than 10⁻⁷ mbar.

Preference is likewise given to an organic electroluminescent device, characterised in that one or more layers are applied by means of the OVPD (organic vapour phase deposition) process or with the aid of carrier-gas sublimation, where the materials are applied at a pressure between 10⁻⁵ mbar and 1 bar. A special case of this process is the OVJP (organic vapour jet printing) process, in which the materials are applied directly through a nozzle and thus structured (for example M. S. Arnold et al., Appl. Phys. Lett. 2008, 92, 053301).

Preference is furthermore given to an organic electroluminescent device, characterised in that one or more layers are produced from solution, such as, for example, by spin coating, or by means of any desired printing process, such as, for example, screen printing, flexographic printing, nozzle printing or offset printing, but particularly preferably LITI (light induced thermal imaging, thermal transfer printing) or ink-jet printing. Soluble compounds of the formula (I) or (II) are necessary for this purpose. High solubility can be achieved by suitable substitution of the compounds.

It is furthermore preferred, for the production of the organic electroluminescent device, for one or more layers to be applied from solution and for one or more layers to be applied by a sublimation process.

The present invention therefore furthermore relates to a process for the production of an organic electroluminescent device according to the invention, characterised in that at least one layer is applied by means of a sublimation process and/or in that at least one layer is applied by means of an OVPD (organic vapour phase deposition) process or with the aid of carrier-gas sublimation and/or in that at least one layer is applied from solution, by spin coating or by means of a printing process.

The following working examples serve for more detailed explanation of the invention and its technical effects and should not be interpreted as being restrictive.

### WORKING EXAMPLES

### A) Determination of HOMO, LUMO, Singlet and Triplet Levels

The HOMO and LUMO energy levels and the energy of the lowest triplet state T₁ and of the lowest excited singlet state S₁ of the materials are determined via quantum-chemical calculations. For this purpose, the

“Gaussian09W” (Gaussian Inc.) program package is used. For the calculation of organic substances without metals (indicated by method “org.” in Table 1), firstly a geometry optimisation is carried out using the “Ground State/Semi-empirical/Default Spin/AM1/Charge 0/Spin Singlet” method, An energy calculation is subsequently carried out on the basis of the optimised geometry. The “TD-SFC/DFT/Default Spin/133PW91” method with the “6-31G(d)” base set is used here (charge 0, spin singlet). For metal-containing compounds (indicated by method “org.-m” in Table 1), the geometry is optimised via the “Ground State/Hartree-FockJDefault Spin/LanL2MB/Charge 0/Spin Singlet” method. The energy calculation is carried out analogously to the organic substances, as described above, with the difference that the “LanL2DZ” base set is used for the metal atom and the “6-31G(d)” base set is used for the ligands. The energy calculation gives the HOMO energy level HEh and LUMO energy level LEh in hartree units. The HOMO and LUMO energy levels calibrated with reference to cyclic voltammetry measurements are determined therefrom in electron volts as follows:

HOMO(eV)=((HEh*27.212)−0.9899)/1.1206

LUMO(eV)=((LEh*27.212)−2,0041)/1.385

These values are to be regarded for the purposes of this application as the HOMO and LUMO energy levels of the materials respectively.

The lowest triplet state T₁ is defined as the energy of the triplet state having the lowest energy which arises from the quantum-chemical calculation described.

The lowest excited singlet state Si is defined as the energy of the excited singlet state having the lowest energy which arises from the quantum-chemical calculation described.

Table 1 shows the HOMO and LUMO energy levels and S₁ and T₁ of he various materials.

TABLE 1
HOMO, LUMO, T₁, S₁ of the relevant materials

| | | HOMO | LUMO | S₁ | T₁ |
|---|---|---|---|---|---|
| Material | Method | (eV) | (eV) | (eV) | (eV) |
| D1 | Org. | −6.11 | −3.40 | 2.50 | 2.41 |

### B) Determination of the PL Quantum Efficiency (PLQE)

A 50 nm thick film of the emission layers used in the various OLEDs is applied to a suitable transparent substrate, preferably quartz, i.e. the layer comprises the same materials in the same concentration as in the OLED. The same production conditions as in the production of the emission layer for the OLEDs are used here. An absorption spectrum of this film in the wavelength range 350-500 nm is measured. To this end, the reflection spectrum R (λ) and the transmission spectrum T (λ) of the sample are determined at an angle of incidence of 6° (i.e. virtually perpendicular incidence). For the purposes of this application, the absorption spectrum is defined as A(λ)=1−R(λ)−T(λ).

If A(λ)≦0.3 in the range 350-500 nm, the wavelength belonging to the maximum of the absorption spectrum in the range 350-500 nm is defined as λ_{exc}. If A(λ)>0.3 for any wavelength, λ_{exc} is defined as the greatest wavelength at which A(λ) changes from a value less than 0.3 to a value greater than 0.3 or from a value greater than 0.3 to a value less than 0.3.

The PLQE is determined using a Hamamatsu C9920-02 measurement system. The principle is based on excitation of the sample with light of defined wavelength and measurement of the absorbed and emitted radiation. The sample is located in an Ulbricht sphere (“integrating sphere”) during the measurement. The spectrum of the excitation light is approximately Gaussian, with a haft-value width <10 nm and a peak wavelength λ_{exc} as defined above.

The PLQE is determined by the usual evaluation method for the said measurement system. It must be strictly ensured that the sample does not come into contact with oxygen at any point, since the PLQE of materials having a small energy separation between S₁ and T₁ is very greatly reduced by oxygen (H. Uoyama et al,, Nature 2012, Vol. 492, 234).

Table 2 shows the PLQE for the emission layers of the OLEDs as defined above together with the excitation wavelength used.

### C) Determination of the Decay Time

The decay time is determined using a sample which has been prepared as described above under “Determination of the PL quantum efficiency (PLQE)”. The measurement is carried out in vacuo. The sample is excited at room temperature by a laser pulse of suitable intensity (wavelength 266 nm, pulse duration about 1.5 ns). After the excitation (defined as t=0), the change in the emitted photoluminescence over time is measured. For the measurement data from time t=250 ns, the decay time tₐ=tₑ−250 ns is determined. tₑ here is the time after t=250 ns at which the intensity has for the first time dropped to 1/e of its value at t=250 ns.

### D) Synthesis of Compounds

Most of the compounds used in the following examples are known from the prior art. For example, the synthesis of dopant D1 is disclosed in Uoyama et al., Nature 2012, Vol. 492, 234. The synthesis of host materials H1, H2 and H3 is disclosed in WO 2011/057706, and the synthesis of H4 is disclosed in WO 2012/048266. The synthesis of H5 is disclosed in WO 2012/12145173, the synthesis of H6 is disclosed in US 2009/0030202.

The synthesis of H7 is described below:

### Step 1

### Synthesis of 2-bromo-8-[1,1′;3′,1″]terphenyl-5′-yldibenzo-thiophene

2.47 g (8.1 mmol) of tetrakistriphenylphosphinopalladium(0) are added to a vigorously stirred suspension of 15 g (40 mmol) of 2,8-dibromodibenzo-thiophene, 11 g (40 mmol) of (3,5-diphenylphenyl)boronic acid and 63.9 g (127 mmol) of Na₂CO₃ in 500 ml of DMF, and the mixture is subsequently heated under reflux for 16 h. After cooling, the precipitated solid is filtered off with suction, washed three times with 50 ml of toluene, three times with 50 ml of ethanol : water (1:1, v:v) and three times with 100 ml of ethanol. The product is then recrystallised three times from DMF (about 15 ml g), giving 12.5 g (25 mmol) of product (88.0% of theory) in a purity of 99.9% (HPLC).

### Step 2

### Synthesis of 2-(8-[1,1′;3′,1″]terphenyl-5′-yidibenzothiophen-2-yl)boronic acid

52 ml (30 mmol) of n-butyllithium (2.5 M in n-hexane) are added dropwise to a suspension of 49 g (100 mmol) of 2-bromo-8-[1,1′;3′,1″]terphenyl-5′-yl-dibenzothiophene in 1000 ml of THF at -78° C. with vigorous stirring, and the mixture is stirred for a further 2 h. 16.7 ml (150 mmol) of trimethyl borate are added to the red solution in one portion with vigorous stirring, the mixture is stirred at −78° C. for a further 30 min., then warmed to room temperature over the course of 3 h, 300 ml of water are added, and the mixture is stirred for 30 min. The organic phase is separated off and evaporated to dryness in vacuo. The solid is taken up in 100 ml of n-hexane, filtered off with suction, washed once with 100 ml of hexane and dried in vacua Yield: 37 g (81 mmol), 83%, purity about 90% (NMR) of boronic acid, with varying amounts of boronic anhydride and boronic acid. The boronic acid can be employed in the next step without further purification.

### Step 3

### Synthesis of 8,8R-bis-[1,1′;3′,1″]terphenyl-5′-yl[2,2′]bi[dibenzo-thiophenyl]

66.7 g (136 mmol) of 2-bromo-8-[1,1′;3′1″]terphenyl-5′-yldibenzothiophene, 65.6 g (144 mmol) of 2-(8-[1,1′;3′1″]terphenyl-5′-yldibenzothiophen-2-yl)-boronic acid and 78.9 ml (158 mmol) of Na₂CO₃ (2M solution) are suspended in 120 ml of toluene, 120 ml of ethanol and 100 ml of water. 2.6 g (2.2 mmol) of Pd(PPh₃)₄ are added to this suspension, and the reaction mixture is heated under reflux for 16 h. After cooling, the organic phase is separated off, filtered through silica gel, washed three times with 200 ml of water and subsequently evaporated to dryness. The residue is recrystallised from toluene and finally sublimed in a high vacuum (p=5×10⁻⁵ mbar). The yield is 105 g (130 mmol), corresponding to 96% of theory.

### E) Production of OLEDs

The data of various OLEDs are presented in Examples V1 and E1 to E7 below (see Tables 1 and 2).

Glass plates coated with structured ITO (indium tin oxide) in a thickness of 50 nm form the substrates for the OLEDs. The substrates are wet-cleaned (dishwasher, Merck Extran detergent), subsequently dried by heating at 250° C. for 15 min and treated firstly with an oxygen plasma and then with an argon plasma before the coating.

The OLEDs have in principle the following layer structure: substrate/hole-injection layer (HIL)/hole-transport layer (HTL) interlayer (IL)/electron-blocking layer (EBL)/emission layer (EML)/hole-blocking layer (HBL) electron-transport layer (ETL) and finally a cathode. The cathode is formed by an aluminium layer with a thickness of 100 nm. The precise structure of the OLEDs is shown in Table 1. The materials required for the production of the OLEDs are shown in Table 3.

All materials are applied by thermal vapour deposition in a vacuum chamber. The emission layer here always consists of a matrix material (host material) and the emitting compound, which is in the form of a dopant. This is admixed with the matrix material in a certain proportion by volume by co-evaporation, An expression such as H1:D1 (95%:5%) here means that material H1 is present in the layer in a proportion by volume of 95% and D1 is present in the layer in a proportion of 5%. Analogously, the electron-transport layer consists of a mixture of two materials.

The OLEDs are characterised by standard methods. For this purpose, the electroluminescence spectra, the current efficiency (measured in cd/A), the power efficiency (measured in lm/W) and the external quantum efficiency (EQE, measured in per cent) as a function of the luminous density, calculated from current/voltage/luminous density characteristic lines (IUL characteristic lines) assuming Lambert emission characteristics, and the life-time are determined, The electroluminescence spectra are determined at a luminous density of 1000 cd/m², and the CIE 1931 x and y colour coordinates are calculated therefrom. The term U1000 in Table 2 denotes the voltage required for a luminous density of 1000 cd/m². CE1000 and PE1000 denote the current and power efficiency respectively which are achieved at 1000 cd/m², Finally, EQE1000 denotes the external quantum efficiency at an operating luminous density of 1000 cd/m2.

The roll-off is defined as EQE at 5000 cd/m² divided by EQE at 500 cd/m², i.e. a high value corresponds to a small drop in the efficiency at high luminous densities, which is advantageous.

The lifetime LT is defined as the time after which the luminous density drops from the initial luminous density to a certain proportion L1 on operation at constant current. A specification of j0=10mAlcm², L1=80% in Table 2 means that the luminous density drops to 80% of its initial value after time LT on operation at 10 mA/cm².

The emitting dopant employed in the emission layer is compound D1, which has an energy separation between S₁ and T₁ of 0.09 eV.

The data of the various OLEDs are summarised in Table 2. Example V1 is a comparative example which comprises compound CBP in accordance with the prior art as matrix material. Examples E1-E7 show data of OLEDs according to the invention which comprise compounds of the formula (I) or (II) as matrix materials.

The measured performance data of the OLEDs show by way of example that excellent values for voltage, efficiency, roll-off and lifetime of the OLEDs are obtained with a structure of the emitting layer in accordance with the present application. The performance data are typically better than those obtained with a structure of the emitting layer in accordance with the prior art (cf. Uoyama et al., Nature 2012, Vol. 492, 234), in which CBP is used as matrix material of the emitting layer.

In particular, excellent values for the operating voltage U1000, the external quantum efficiency EQE, the lifetime and the roll-off are obtained (Table 2).

TABLE 1
Structure of the OLEDs

| | HIL | HTL | IL | EBL | EML | HBL | ETL |
|---|---|---|---|---|---|---|---|
| Ex. | Thickness | Thickness | Thickness | Thickness | Thickness | Thickness | Thickness |
| V1 | HAT | SpA1 | HAT | SpMA1 | CBP:D1 | IC1 | ST2:LiQ |
| | 5 nm | 70 nm | 5 nm | 20 nm | (95%:5%) | 10 nm | (50%:50%) |
| | | | | | 15 nm | | 40 nm |
| E1 | HAT | SpA1 | HAT | SpMA1 | H1:D1 | IC1 | ST2:LiQ |
| | 5 nm | 70 nm | 5 nm | 20 nm | (95%:5%) | 10 nm | (50%:50%) |
| | | | | | 15 nm | | 40 nm |
| E2 | HAT | SpA1 | HAT | SpMA1 | H2:D1 | IC1 | ST2:LiQ |
| | 5 nm | 70 nm | 5 nm | 20 nm | (95%:5%) | 10 nm | (50%:50%) |
| | | | | | 15 nm | | 40 nm |
| E3 | HAT | SpA1 | HAT | SpMA1 | H3:D1 | IC1 | ST2:LiQ |
| | 5 nm | 70 nm | 5 nm | 20 nm | (95%:5%) | 10 nm | (50%:50%) |
| | | | | | 15 nm | | 40 nm |
| E4 | HAT | SpA1 | HAT | SpMA1 | H4:D1 | IC1 | ST2:LiQ |
| | 5 nm | 70 nm | 5 nm | 20 nm | (95%:5%) | 10 nm | (50%:50%) |
| | | | | | 15 nm | | 40 nm |
| E5 | HAT | SpA1 | HAT | SpMA1 | H5:D1 | IC1 | ST2:LiQ |
| | 5 nm | 70 nm | 5 nm | 20 nm | (95%:5%) | 10 nm | (50%:50%) |
| | | | | | 15 nm | | 40 nm |
| E6 | HAT | SpA1 | HAT | SpMA1 | H6:D1 | IC1 | ST2:LiQ |
| | 5 nm | 70 nm | 5 nm | 20 nm | (95%:5%) | 10 nm | (50%:50%) |
| | | | | | 15 nm | | 40 nm |
| E7 | HAT | SpA1 | HAT | SpMA1 | H7:D1 | IC1 | ST2:LiQ |
| | 5 nm | 70 nm | 5 nm | 20 nm | (95%:5%) | 10 nm | (50%:50%) |
| | | | | | 15 nm | | 40 nm |

TABLE 2
Data of the OLEDs

| | U1000 | CE1000 | PE1000 | EQE | CIE x/y at | Roll- | | L1 | LT | PLQE | λ_{exc} |
|---|---|---|---|---|---|---|---|---|---|---|---|
| Ex. | (V) | (cd/A) | (lm/W) | 1000 | 1000 cd/m² | off | L0:j0 | % | (h) | % | nm |
| V1 | 4.2 | 44 | 33 | 14.1% | 0.25/0.58 | 0.60 | 10 mA/cm² | 80 | 23 | 100 | 350 |
| E1 | 3.7 | 47 | 41 | 15.0% | 0.31/0.58 | 0.65 | 10 mA/cm² | 80 | 38 | 65 | 350 |
| E2 | 3.6 | 52 | 46 | 16.3% | 0.33/0.58 | 0.70 | 10 mA/cm² | 80 | 32 | 70 | 350 |
| E3 | 3.5 | 44 | 40 | 13.7% | 0.32/0.58 | 0.67 | 10 mA/cm² | 80 | 57 | 62 | 350 |
| E4 | 3.9 | 39 | 31 | 12.8% | 0.33/0.57 | 0.62 | 10 mA/cm² | 80 | 43 | 74 | 350 |
| E5 | 3.9 | 50 | 40 | 15.3% | 0.30/0.58 | 0.64 | 10 mA/cm² | 80 | 21 | 82 | 350 |
| E6 | 4.1 | 45 | 35 | 14.0% | 0.28/0.57 | 0.62 | 10 mA/cm² | 80 | 33 | 94 | 350 |
| E7 | 4.4 | 39 | 28 | 12.2% | 0.28/0.57 | 0.58 | 10 mA/cm² | 80 | 48 | 77 | 350 |

```
TABLE 3
Structural formulae of the materials for the OLEDs
  HAT
  SpA1
  ST2
  SpMA1
  D1
  LiQ
  CBP (prior art)
  IC1
  H1
  H2
  H3
  H4
  H5
  H6
  H7
```

1-18. (canceled)

19. An organic electroluminescent device comprising an emitting layer comprising a compound E and a compound M, wherein compound E has a difference in value between the energies of the S₁ and T₁ states of at most 0.15 eV, and wherein compound M is a compound of formulae (I) or (II):
wherein
Y is on each occurrence, identically or differently, O, S, or Se;
Z is on each occurrence, identically or differently, CR¹, C, or N, wherein Z is equal to C if a group L¹ or L² is bonded to Z together with the group bonded thereto;
L¹ and L² are on each occurrence, identically or differently, a single bond or a divalent group;
Ar¹ is on each occurrence, identically or differently, an aromatic or heteroaromatic ring system having 5 to 40 aromatic ring atoms, which is optionally substituted by one or more radicals R²;
R¹ and R² are on each occurrence, identically or differently, H, D, F, C(═O)R³, CN, Si(R³)₃, N(R³)₂, P(═O)(R³)₂, OR³, S(═O)R³, S(═O)₂R³, a straight-chain alkyl or alkoxy group having 1 to 20 C atoms, a branched or cyclic alkyl or alkoxy group having 3 to 20 C atoms, or an alkenyl or alkynyl group having 2 to 20 C atoms, wherein the above-mentioned groups are optionally substituted by one or more radicals R³ and wherein one or more CH₂ groups in the above-mentioned groups are optionally replaced by —R³C═CR³—, —C≡C—, Si(R³)₂, C═O, C═NR³, —C(═O)O—, —C(═O)NR³13 , NR³, P(═O)(R³), —O—, —S—, SO, or SO₂, or an aromatic or heteroaromatic ring system having 5 to 30 aromatic ring atoms, which is optionally substituted by one or more radicals R³, and wherein two or more radicals R¹ or R² are optionally linked to one another so as to define a ring;
R³ is on each occurrence, identically or differently, H, D, F, C(═O)R⁴, CN, Si(R⁴)₃, N(R⁴)₂, P(═O)(R⁴)₂, OR⁴, S(═O)R⁴, S(═O)₂R⁴, a straight-chain alkyl or alkoxy group having 1 to 20 C atoms, a branched or cyclic alkyl or alkoxy group having 3 to 20 C atoms, or an alkenyl or alkynyl group having 2 to 20 C atoms, wherein the above-mentioned groups are optionally substituted by one or more radicals R⁴ and wherein one or more CH₂ groups in the above-mentioned groups are optionally replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO, or SO₂, or an aromatic or heteroaromatic ring system having 5 to 30 aromatic ring atoms, which is optionally substituted by one or more radicals R⁴, and wherein two or more radicals R³ are optionally linked to one another so as to define a ring;
R⁴ is on each occurrence, identically or differently, H, D, F or an aliphatic, aromatic, or heteroaromatic organic radical having 1 to 20 C atoms, wherein one or more H atoms are optionally replaced by D or F; and wherein two or more substituents R⁴ are optionally linked to one another so as to define a ring;
n is 0 or 1;
wherein the energies are determined by quantum-chemical calculation.

20. The organic electroluminescent device of claim 19, wherein not more than one Z per ring in formulae (I) and (II) is N.

21. The organic electroluminescent device of claim 19, wherein Y in formulae (I) and (II) is on each occurrence, identically or differently, O or S.

22. The organic electroluminescent device of claim 19, wherein L¹ in formula (I) is on each occurrence, identically or differently, a single bond, Si(R²)₂, or an aromatic or heteroaromatic ring system having 5 to 30 aromatic ring atoms, which is optionally substituted by one or more radicals R².

23. The organic electroluminescent device of claim 19, wherein L² in formula (II) is a single bond or an aromatic or heteroaromatic ring system having 5 to 60 aromatic ring atoms, which is optionally substituted by one or more radicals R².

24. The organic electroluminescent device of claim 19, wherein Ar¹ in formula (I) is selected from the group consisting of phenyl, biphenyl, terphenyl, quaterphenyl, fluorenyl, spirobifluorenyl, indenofluorenyl, naphthyl, anthracenyl, phenanthrenyl, pyrenyl, fluoranthenyl, furanyl, benzofuranyl, isobenzofuranyl, dibenzofuranyl, thiophenyl, benzothiophenyl, isobenzothiophenyl, dibenzothiophenyl, pyrrolyl, indolyl, isoindolyl, carbazolyl, indolocarbazolyl, indenocarbazolyl, pyridyl, quinolinyl, isoquinolinyl, acridyl, pyrazolyl, imidazolyl, benzimidazolyl, pyridazyl, pyrimidyl, pyrazinyl, and phenanthrolyl, each of which is optionally substituted by radicals R².

25. The organic electroluminescent device of claim 19, wherein compound M is a compound of formulae (I-1) through (I-4):

26. The organic electroluminescent device of claim 19, wherein compound M is a compound of formulae (II-1) to (II-3)

27. The organic electroluminescent device of claim 19, wherein L² is a single bond or a unit of formula (L2):
wherein
Ar³ is on each occurrence, identically or differently, an arylene or heteroarylene group having 6 to 14 aromatic ring atoms, which is optionally substituted by one or more radicals R²; and
k is equal to 1, 2, 3, or 4.

28. The organic electroluminescent device of claim 19, wherein compound M is the matrix material in the emitting layer, and compound E is the emitting compound in the emitting layer.

29. The organic electroluminescent device of claim 19, wherein the emitting layer consists essentially of compound M and compound E.

30. The organic electroluminescent device of claim 19, wherein the proportion of compound M is between 80% and 99% and the proportion of compound E is between 1 and 20%.

31. The organic electroluminescent device of claim 19, wherein
T₁(M)≧T₁(E)
wherein
T₁(M) is the energy of the T₁ state of compound M; and
T₁(E) is the energy of the T₁ state of compound E.

32. The organic electroluminescent device of claim 19, wherein compound E is a luminescent compound which has a luminescence quantum efficiency of at least 60%.

33. The organic electroluminescent device of claim 19, wherein the difference in value between the energies of the S₁ and T₁ states of compound E is less than or equal to 0.10 eV.

34. The organic electroluminescent device of claim 19, wherein compound E is an aromatic compound which comprises both at least one donor substituent and at least one acceptor substituent.

35. The organic electroluminescent device of claim 19, wherein
LUMO(E)−HOMO(M)>S₁(E)−0.4 eV
wherein
LUMO(E) is the LUMO of compound E;
HOMO(M) is the HOMO of compound M; and
S₁(E) is the first excited singlet state S₁ of compound E; and
the energies LUMO(E) and HOMO(M) are determined by quantum-chemical calculations.

36. A process for producing the organic electroluminescent device of claim 19, comprising applying at least one layer by means of a sublimation process and/or by means of a OVPD process or with the aid of carrier-gas sublimation and/orfrom solution, by spin coating or by means of a printing process.
